(19) United States
(12) Patent Application Publication
(10) Pub. No.: US 2003/0138394 A1
(43) Pub. Date: Jul. 24, 2003

(54) Cosmetic and/or pharmaceutical preparations that contain an extract of the plant argania spinosa

(76) Inventors: Zoubida Charrouf, Rabat (MA); Florence Henry, Villers-les-Nancy (FR); Gilles Pauly, Nancy (FR); Philippe Moser, Essey-les-Nancy (FR)

Correspondence Address: COGNIS CORPORATION, 2500 RENAISSANCE BLVD., SUITE 200, GULPH MILLS, PA, 19406

(21) Appl. No.: 10/258,772

(22) PCT Filed: Apr. 19, 2001
(86) PCT No.: PCT/EP01/04439

(30) Foreign Application Priority Data
Apr. 28, 2000 (EP) 00440119.6

(51) Int. Cl. A61K 35/78; A61K 7/06

(52) U.S. Cl. 424/74; 424/725; 424/776

(57) ABSTRACT

The invention relates to cosmetic and/or pharmaceutical preparations that contain saponins derived from an extract of the plant Argania spinosa.

### FIELD OF THE INVENTION

The invention is in the field of cosmetic care substances and relates to novel hair and skincare compositions, and to the use thereof in cosmetics and pharmacy.

### PRIOR ART

Hair cosmetic preparations, such as, for example, hair lacquers or styling agents, contain, as setting agent, synthetic polymers, preferably of the polyvinylpyrrolidone and vinyl acetate type [cf. U.S. Pat. No. 5,637,296], which attach to keratin fibers and provide them with the desired hold and increased flexural strength. However, it is disadvantageous that these films become very brittle after drying, especially upon repeated application, and damage to the hair may additionally result. Ethoxylated C12- to C20-fatty alcohols often described as hair-setting agents are used in conjunction with nonhalogenated, water-soluble solvents. These solvents dry the hair out in an undesired manner and can additionally cause skin irritations. [WO 94/08554].

Conventional conditioning agents of the cationic surfactant or cationic polymer type are synthetic compounds which are used in quite high concentrations, attach to the hair and lead to the desired effect. Upon washing out, these conditioning agents can often not be readily removed, which, in the case of long-term application, may lead to damage to the hair and the scalp.

The desire for hair cosmetic compositions which simultaneously increase the flexural strength of the hair, increase the shine and the volume, but do not lead to damage of the hair upon repeated application is expressed more and more often by the consumer. In addition, there is a growing interest for novel and effective substances for cosmetics which are natural in origin and which can be obtained from renewable raw materials. In addition to good conditioning, these substances should, as far as possible, also have a care effect for the hair and for the skin.

Cosmetic preparations are available nowadays to the consumer in a large number of combinations. Nevertheless, in the market there is the need for products with an improved performance spectrum. In this connection, skin compatibility and the use of natural products are demanded by the customer. In addition, it is desirable to obtain significantly better products by combining active ingredients which are already known, or by discovering new fields of use for classes of substance which are already known. Extracts of plants and ingredients thereof in particular are being used more and more frequently in cosmetics and pharmacy. However, many plants and their potential effects have still not been discovered and many new fields of use for classes of substance which are already known are time and again surprising.

For a long time it has been known that many saponins, which are obtained from a very wide variety of plants and microorganisms, exhibit an anti-free-radical, analgesic and also anti-inflammatory effect. This effect was demonstrated by Alaoui et al. also for the saponins isolated from Argania spinosa [Alaoui K. et al.; Annales pharmaceutique francaises, 1998, 56, 220-228]. For some saponins, an antibiotic and a fungistatic effectiveness has also been found. Saponins, specifically the triterpene saponins, are constructed from a tetra- or pentacyclic triterpene aglycone and one or two glycosidically bonded sugar chains.

Due to their marked foam stability and due to the emulsifying behavior, saponins are usually used in surfactant mixtures [DE 220448].

Saponins from extracts of the plant Hedera helix in combination with extracts from Arnica montana and extracts from colanut are used in cosmetic compositions for the skin which exhibit a slimming effect [DE 3204370]. The only possible use of this combination of two or more extracts and saponins from the plants mentioned is their use as slimming agents with anticellulite action.

### DESCRIPTION OF THE INVENTION

The object of the present patent application was to find plant extracts which can be used in hair treatment and haircare and which, on the basis of their properties, increase or improve, for example, the shine, the combability in the dry and wet state and the volume.

A further object of the present patent application was to provide cosmetic and or pharmaceutical preparations which comprise active ingredients from renewable raw materials and at the same time can be used diversely as care agents in cosmetics both in skincare and also in skin cosmetics.

The invention provides cosmetic and/or pharmaceutical preparations which comprise saponins extracted from the plant Argania spinosa. Surprisingly, it has been found that through the use of saponins from extracts of the plant Argania spinosa, products are obtained which simultaneously have good care and protecting properties for hair and skin, and have high skin compatibility. The compositions obtained in this way are characterized by particularly good effects in the case of hair. They improve the combability of dry and wet hair, increase the shine and volume and at the same time produce increased flexural strength on the hair, which permits the use in combined preparations, such as, for example, daily application of hair-setting agents to wet and also to bleached or permanently waved, damaged hair.

Moreover, it has been found that the compositions obtained in this way can be used particularly readily in skincare as slimming agents with anticellulite action. This comparable action with the saponins from Hedera helix, however, does not require the stimulating effect of further plant extracts from other plants, as is described in DE 3204370 and thus exhibits a decisive advantage over the compositions described therein.

These multiple fields of use of the compositions according to the invention from the renewable raw material of the plant Argania spinosa makes it very attractive for the market and for the consumer. The complex object of the invention was thus achieved through the use of the saponins from Argania spinosa.

For the purposes of the present application, the term “plant” is to be understood as meaning either whole plants, or else parts of plants (leaves, roots, stem, bark, flowers, fruits, fruit flesh and seeds), and also mixtures thereof. For the purposes of the invention, for the extraction of the saponins, particular preference is given to the seeds of the fruit of this plant, in particular to the extraction of the residue from the defatted seeds.

Argania spinosa

The extracts to be used according to the invention are obtained from plants of the family of Sapotaceae, specifically from Argania spinosa. This plant is a tree reminiscent of the olive tree which is to be found primarily in Morocco on the west side of the Atlas mountain range. On its knarled branches and thorny twigs it forms berries of the size and shape of olives with one to two seeds. The oil from the seeds, which has a nut-like taste, is used inter alia as a food oil.

Saponins

For the purposes of the invention, saponins are in principle understood as meaning all saponins which can be isolated from the plant Argania spinosa.

From the residue which is produced during the oil production from the seeds of Argania spinosa, saponins are obtained which differ in their structure from saponins from other plants [Charrouf Z., et al.; Phytochemistry, 1992, 31; 2079-2086]. The saponins here have the names arganine A, arganine B, arganine C, arganine D, arganine E, arganine F and misaponine A. From the stem of the plant it is possible [lacuna] the saponins arganine G, arganine H and arganine J which can be used [Oulad-Ali A., et al.; J. Nat. Prod.; 1996, 59, 193-195]. The aglycone of these saponins has the structure (I) depicted below, said saponins differing in each case in the sugar units at R1 and R3 or by a hydroxyl group at R2. R3 is a tetrasaccharide and R1 is in each case a mono- or disaccharide (e.g. 1,6-diglucose for arganine A and B).

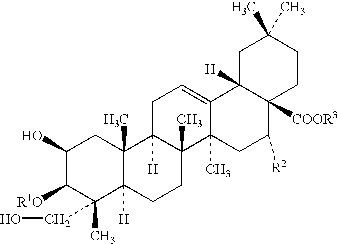

The saponins according to the invention exhibit low toxicity in toxicological test [sic] on mice and rats [Alaoui K., et al.; Ann. Pharmaceutiques francaises, 1998, 5, 213-219]. Compared with other saponins, such as, for example, from Gypsophila paniculata, the inventors were also able to demonstrate a significantly lower toxicity through tests on human fibroblasts.

The saponins to be used according to the invention correspond to arganine A, arganine B, arganine C, arganine C [sic], arganine D, arganine E, arganine F, misaponine A, and also arganine G, arganine H and arganine J. They can be used as a mixture of two or more, or as pure substance in the cosmetic and or pharmaceutical preparation. Particular preference is given to mixtures of arganine A, arganine B, arganine C, arganine C [sic] , arganine D, arganine E, arganine F, misaponine A, where the proportions of the saponins in the mixtures can vary.

Extraction

The extracts to be used according to the invention are prepared by customary methods of extraction of plants or parts of plants. With regard to the suitable conventional extraction methods, such as maceration, remaceration, digestion, agitation maceration, fluidized-bed extraction, ultrasound extraction, countercurrent extraction, percolation, repercolation, evacolation (extraction under reduced pressure), diacolation and solid-liquid extraction under continuous reflux which is carried out in a Soxhlet extractor, each of which is known to the person skilled in the art and any of which can be used in principle, reference may be made by way of example to Hagers Handbuch der Pharmazeutischen Praxis, (5^{th} edition, Vol. 2, pp. 1026-1030, Springer Verlag, Berlin-Heidelberg-N.Y. 1991). Starting material which may be used are fresh or dried plants or parts of plants, although usually the starting materials are defatted plants and/or parts of plants which can be mechanically comminuted prior to extraction. In this connection, all comminution methods known to the person skilled in the art are suitable, trituration with a mortar being given by way of example. In a particular embodiment, the extracts used are obtained by extraction of the stem, the roots, the leaves, the flowers or the fruits. Particular preference is given to the extraction of the seeds.

Solvents which can be used for carrying out the extractions are preferably organic solvents, water or mixtures of organic solvents and water, in particular low molecular weight alcohols, esters, ketones or halogen-containing hydrocarbons with greater or lesser water contents (distilled or undistilled), preferably aqueous, alcoholic solutions of a temperature greater than 20° C. (referred to below as room temperature). Particular preference is given to the extraction with water, methanol, ethanol, acetone, propylene glycols, polyethylene glycols, ethyl acetate, dichloromethane, trichloromethane, and mixtures thereof. The extraction usually takes place at 20 to 100° C., preferably at 20 to 85° C., in particular at room temperature. In one possible embodiment, the extraction is carried out under an inert gas atmosphere to avoid oxidation of the ingredients of the extract. The extraction times are adjusted by the person skilled in the art depending on the starting material, the extraction method, the extraction temperature, the ratio of solvent to raw material, etc. After the extraction, the resulting crude extracts can optionally be subjected to further customary steps, such as, for example, purification, concentration and/or decoloration. If desired, the extracts prepared in this way can, for example, be subjected to selective removal of individual undesired ingredients. The extraction can be carried out to any desired degree of extraction, but is usually carried out exhaustively. Typical yields (=dry substance amount of the extract based on amount of raw material used) during the extraction of dried plants or dried parts of plants, optionally defatted, are in the range from 3 to 20% by weight, in particular 4 to 16% by weight. The present invention encompasses the finding that the extraction conditions and also the yields of the end extracts can be chosen depending on the desired field of use. If desired, the extracts can then be subjected, for example, to spray- or freeze-drying.

According to the invention, the extracts of this plant comprise 10 to 100% by weight of saponins, preferably 20 to 70% by weight, in particular 30 to 50% by weight.

The amount of plant extracts used in said preparations is governed by the concentration of the individual ingredients and by the type of applications of the extracts. The total amount of the plant extract which is present in the preparations according to the invention is usually 0.01 to 25% by weight, preferably 0.03 to 5% by weight, in particular 0.03 to 0.4% by weight, based on the final preparation, with the proviso that the quantitative amounts add up to 100% by weight with water and optionally further auxiliaries and additives.

The total content of auxiliaries and additives may be 1 to 50% by weight, preferably 5 to 40% by weight, based on the final preparation of the cosmetic and/or pharmaceutical preparations. The preparations can be prepared by customary cold or hot processes; preference is given to using the phase-inversion temperature method.

For the purposes of the invention, active substance refers to the proportion of substances and also auxiliaries and additives which are present in the cosmetic composition, with the exception of the additionally added water.

A particular embodiment of the present invention relates to the diverse use of the saponin-containing plant extracts from Argania spinosa, for example

in care compositions for hair and/or skin, in particular against stress and damaging environmental influences

in care compositions for improving the combability, the shine, the volume and the flexural strength of hair

as care composition with slimming and anticellulite properties for the skin

as skincare composition with antirosacea effect;

in protective and restorative care compositions for stimulating the metabolism and the immune response of the skin and of the hair follicles with revitalizing and reactivating activities for the skin and the hair follicles;

in sunscreens, in particular against the damage of fibroblasts and/or keratinocytes by UVA radiation and/or UVB radiation.

Care Compositions

For the purposes of the invention, care compositions are understood as meaning care compositions for hair and skin. These care compositions include, inter alia, cleansing and restorative action for hair and skin.

The aim of haircare is to maintain the natural condition of freshly grown hair for as long as possible, or to restore it in cases of damage. Characteristics of natural healthy hair are silky shine, low porosity, vigorous coupled with soft fullness and pleasant smooth feel (good “hand”). The care compositions according to the invention have a smoothing effect on the hair, they improve the combability and improve volume and shine. The use of the care compositions according to the invention increases the flexural strength of the hair, so that the natural vigor of the hair is increased or the vigor of damaged hair is restored.

Moreover, the preparations according to the invention exhibit an excellent skincare effect coupled with high skin compatibility. In addition, they exhibit good stability, in particular against oxidative decomposition of the products.

These care compositions include slimming and anticellulite properties for the skin. In addition, for the purposes of the invention, care compositions are understood as meaning those which are used in cases of skin reddening, such as rosacea, as the result of e.g. infections or as a result of allergies, and bring about a reduction in skin reddening. In principle, the extracts according to the invention can be used in all cosmetic products.

In addition, the preparations according to the invention exhibit protective and restorative activity by stimulating the metabolism and the immune response of the skin and of the hair follicles with revitalizing and reactivating activities for the skin and the hair follicles. As a result of this activity, a use as skin rejuvenating composition, in particular, is possible.

In a particular embodiment, the extracts according to the invention are used in sunscreens. In particular, they exhibit activity against UVB radiation on keratinocytes and against UVA radiation on fibroblasts and are preferably used against the damage on skin cells by UVB and/or UVA radiation.

Examples of cosmetic products are described in their formulations in the Tables 7 to 10.

### INDUSTRIAL APPLICABILITY

Cosmetic and/or Pharmaceutical Preparations

The preparations according to the invention can be used for the preparation of cosmetic and/or pharmaceutical preparations, such as, for example, hair shampoos, hair lotions, foam baths, shower preparations, creams, gels, lotions, alcoholic and aqueous/alcoholic solutions, emulsions, wax/fatty compositions, stick preparations, powders or ointments. These compositions can also comprise, as further auxiliaries and additives, mild surfactants, oily bodies, emulsifiers, pearlescent waxes, bodying agents, thickeners, superfatting agents, stabilizers, polymers, silicone compounds, fats, waxes, lecithins, phospholipids, biogenic active ingredients, UV light protection factors, antioxidants, deodorants, antiperspirants, antidandruff agents, film formers, swelling agents, insect repellents, self-tanning agents, tyrosine inhibitors (depigmentation agents), hydrotropes, solubilizers, preservatives, perfume oils, dyes and the like.

Surfactants

Surface-active substances which may be present are anionic, nonionic, cationic and/or amphoteric or amphoteric [sic] surfactants, the content of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular wheat-based vegetable products) and alkyl (ether) phosphates. If the anionic surfactants contain polyglycol ether chains, these may have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. If the nonionic surfactants contain polyglycol ether chains, these may have a conventional homolog distribution, but preferably have a narrowed homolog distribution. Typical examples of cationic surfactants are quaternary ammonium compounds, such as, for example, dimethyldistearylammonium chloride, and ester quats, in particular quaternized fatty acid trialkanolamine ester salts. Typical examples of amphoteric or zwitterionic surfactants are alkylbetaines, alkylamidobetaines, aminopropionates, aminoglycinates, imidazolinium-betaines and sulfobetaines. Said surfactants are exclusively known compounds. With regard to structure and preparation of these substances, reference may be made to relevant review works, for example, J. Falbe (ed.), “Surfactants in Consumer Products”, Springer Verlag, Berlin, 1987, pp. 54-124 or J. Falbe (ed.), “Katalysatoren, Tenside und Mineralöladditive”, Thieme Verlag, Stuttgart, 1978, pp. 123-217. Typical examples of particularly suitable mild, i.e. particularly skin-compatible surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines, amphoacetals and/or protein fatty acid condensates, the latter preferably based on wheat proteins.

Oily Bodies

Suitable oily bodies are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkylhydroxycarboxylic acids with linear or branched C₆-C₂₂-fatty alcohols (cf. DE 19756377 A1), in particular dioctyl malates, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonates (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsoly® TN.), linear or branched, symmetrical or unsymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicon methicone types, inter alia) and/or aliphatic or naphthenic hydrocarbons, such as, for example, such as [sic] squalane, squalene or dialkylcyclohexanes.

Emulsifiers

Suitable emulsifiers are, for example, nonionogenic surfactants from at least one of the following groups:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and onto alkylamines having 8 to 22 carbon atoms in the alkyl radical;

alkyl and/or alkenyl oligoglycosides having 8 to 22 carbon atoms in the alk(en)yl radical and the ethoxylated analogs thereof;

addition products of from 1 to 15 mol of ethylene oxide to castor oil and/or hydrogenated castor oil;

addition products of from 15 to 60 mol of ethylene oxide to castor oil and/or hydrogenated castor oil;

partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide;

partial esters of polyglycerol (average degree of self-condensation 2 to 8), polyethylene glycol (molecular weight 400 to 5 000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;

mixed esters of pentaerythritol, fatty acids, citric acid and fatty alcohol as in German Patent 1165574 and/or mixed esters of fatty acids having 6 to 22 carbon atoms, methylglucose and polyols, preferably glycerol or polyglycerol,

mono-, di- and trialkyl phosphates, and mono-, di- and/or tri-PEG alkyl phosphates and salts thereof;

wool wax alcohols;

polysiloxane-polyalkyl-polyether copolymers and corresponding derivatives;

block copolymers, e.g. polyethylene glycol-30 dipolyhydroxystearates;

polymer emulsifiers, e.g. Pemulen grades (TR-1, TR-2) from Goodrich;

polyalkylene glycols, and

glycerol carbonate.

The addition products of ethylene oxide and/or of propylene oxide onto fatty alcohols, fatty acids, alkylphenols or onto castor oil are known, commercially available products. These are homolog mixtures whose average degree of alkoxylation corresponds to the ratio of the amounts of ethylene oxide and/or propylene oxide and substrate with which the addition reaction is carried out. C_{12/18}-fatty acid mono- and diesters of addition products of ethylene oxide onto glycerol are known from German Patent 2024051 as refatting agents for cosmetic preparations.

Alkyl and/or alkenyl oligoglycosides, their preparation and their use are known from the prior art. They are prepared, in particular, by reacting glucose or oligosaccharides with primary alcohols having 8 to 18 carbon atoms. With regard to the glycoside radical, both monoglycosides, in which a cyclic sugar radical is glycosidically bonded to the fatty alcohol, and also oligomeric glycosides having a degree of oligomerization of up to, preferably, about 8, are suitable. The degree of oligomerization here is a statistical average value which is based on a homolog distribution customary for such technical-grade products.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid moglyceride [sic], ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride, and the technical-grade mixtures thereof which may also comprise small amounts of triglyceride as a minor product of the preparation process. Likewise suitable are addition products of 1 to 30 mol, preferably 5 to 10 mol, of ethylene oxide to said partial glycerides.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Likewise suitable are addition products of 1 to 30 mol, preferably 5 to 10 mol, of ethylene oxide to said sorbitan esters.

Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, zwitterionic surfactants can be used as emulsifiers. The term “zwitterionic surfactants” refers to those surface-active compounds which carry at least one quaternary ammonium group and at least one carboxylate and one sulfonate group in the molecule. Particularly suitable zwitterionic surfactants are the betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethyl-carboxymethyl glycinate. Particular preference is given to the fatty acid amide derivative known under the CTFA name Cocamidopropyl Betaine. Likewise suitable emulsifiers are ampholytic surfactants. The term “ampholytic surfactants” means those surface-active compounds which, apart from a C_{8/18}-alkyl or -acyl group in the molecule, contain at least one free amino group and at least one —COOH or —SO₃H group and are capable of forming internal salts. Examples of suitable ampholytic surfactants are N-alkylglycines, N-alkylpropionic [sic] acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkyl aminopropionate, cocoacylaminoethyl aminopropionate and C_{12/18}-acylsarcosine. Finally, cationic surfactants are also suitable emulsifiers, those of the ester quat type, preferably methyl-quaternized difatty acid triethanolamine ester salts, being particularly preferred.

Fats and Waxes

Typical examples of fats are glycerides, i.e. solid or liquid vegetable or animal products which consist essentially of mixed glycerol esters of higher fatty acids, suitable waxes are inter alia natural waxes, such as, for example, candelilla wax, carnauba wax, japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), such as, for example, montan ester waxes, sasol waxes, hydrogenated jojoba waxes, and synthetic waxes, such as, for example, polyalkylene waxes and polyethylene glycol waxes. In addition to the fats, suitable additives are also fat-like substances, such as lecithins and phospholipids. The term lecithins is understood by the person skilled in the art as meaning those glycerophospholipids which form from fatty acids, glycerol, phosphoric acid and choline by esterification. Lecithins are thus frequently also [lacuna] as phosphatidylcholines (PC). Examples of natural lecithins which may be mentioned are the cephalins, which are also referred to as phosphatidic acids and represent derivatives of 1,2-diacyl-sn-glycerol-3-phosphoric acids. By contrast, phospholipids are usually understood as meaning mono- and, preferably, diesters of phosphoric acid with glycerol (glycerophosphates), which are generally considered to be fats. In addition, sphingosines and sphingolipids are also suitable.

Pearlescent Waxes

Examples of suitable pearlescent waxes are: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Bodying Agents and Thickeners

Suitable bodying agents are primarily fatty alcohols or hydroxy fatty alcohols having 12 to 22, and preferably 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, Aerosil grades (hydrophilic silicas), polysaccharides, in particular xanthan gum, guar guar, agar agar, alginates and Tyloses, carboxymethylcellulose and hydroxyethylcellulose, and also relatively high molecular weight polyethylene glycol mono- and diesters of fatty acids, polyacrylates (e.g. Carbopols® and Pemulen grades from Goodrich; Synthalens® from Sigma; Keltrol grades from Kelco; Sepigel grades from Seppic; Salcare grades from Allied Colloids), polyacrylamides, polymers, polyvinyl alcohol and polyvinylpyrrolidone, surfactants, such as, for example, ethoxylated fatty acid glycerides, esters of fatty acids with polyols such as, for example, pentaerythritol or trimethylolpropane, fatty alcohol ethoxylates having a narrowed homolog distribution or alkyl oligoglucosides, and electrolytes such as sodium chloride and ammonium chloride.

Superfatting Agents

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter also serving as foam stabilizers.

Stabilizers

Stabilizers which can be used are metal salts of fatty acids, such as, for example, magnesium, aluminum and/or zinc stearate or ricinoleate.

Polymers

Suitable cationic polymers are, for example, cationic cellulose derivatives, such as, for example, a quaternized hydroxyethylcellulose obtainable under the name Polymer JR 400® from Amerchol, cationic starch, copolymers of diallylammonium salts and acrylamides, quaternized vinylpyrrolidone-vinylimidazole polymers, such as, for example, Luviquat® (BASF), condensation products of polyglycols and amines, quaternized collagen polypeptides, such as, for example, lauryldimonium hydroxypropyl hydrolyzed collagen (Lamequat® L/Grünau), quaternized wheat polypeptides, polyethyleneimine, cationic silicone polymers, such as, for example, amodimethicones, copolymers of adipic acid and dimethylaminohydroxypropyldiethylenetriamine (Cartaretins®/Sandoz), copolymers of acrylic acid with dimethyldiallylammonium chloride (Merquat® 550/Chemviron), polyaminopolyamides, as described, for example, in FR 2252840 A, and crosslinked water-soluble polymers thereof, cationic chitin derivatives, such as, for example, quaternized chitosan, optionally in microcrystalline dispersion, condensation products from dihaloalkyls, such as, for example, dibromobutane with bisdialkylamines, such as, for example, bis-dimethylamino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamido-propyltrimethylammonium chloride-acrylate copolymers, octylacrylamide-methyl methacrylate-tert-butylamino-ethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones. Further suitable polymers and thickeners are listed in Cosm. Toil. 108, 95 (1993).

Silicone Compounds

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethylsiloxane units and hydrogenated silicates. A detailed review of suitable volatile silicones can additionally be found in Todd et al., Cosm. Toil. 91, 27 (1976).

UV Light Protection Filters and Antioxidants

UV light protection factors are, for example, to be understood as meaning organic substances (light protection filters) which are liquid or crystalline at room temperature and which are able to absorb ultra-violet rays and give off the absorbed energy again in the form of longer-wavelength radiation, e.g. heat. UVB filters can be oil-soluble or water-soluble. Examples of oil-soluble substances are:

3-benzylidenecamphor or 3-benzylidenenorcamphor and derivatives thereof, e.g. 3-(4-methylbenzylidene)-camphor, as described in EP 0693471 B1;

4-aminobenzoic acid derivatives, preferably 2-ethylhexyl 4-(dimethylamino)benzoate, 2-octyl 4-(dimethylamino)benzoate and amyl 4-(dimethylamino)-benzoate;

esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenyl-cinnamate (octocrylene);

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di-2-ethylhexyl 4-methoxybenzmalonate [sic];

triazine derivatives, such as, for example, 2,4,6-trianilino(p-carbo-2′-ethyl-1′-hexyloxy)-1,3,5-triazine and octyltriazone, as described in EP 0818450 A1 or dioctylbutamidotriazone (Uvasorb® HEB);

propane-1,3-diones, such as, for example, 1-(4-tert-butylphenyl)-3-(4′-methoxyphenyl)propane-1,3-dione;

ketotricyclo(5.2.1.0)decane derivatives, as described in EP 0694521 B1.

Suitable water-soluble substances are:

2-phenylbenzimidazole-5-sulfonic acid and the alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)-benzenesulfonic acid and 2-methyl-5-(2-oxo-3-bornylidene)sulfonic acid and salts thereof.

Suitable typical UV-A filters are, in particular, derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF). The UV-A and UV-B filters can of course also be used in mixtures. Particularly favorable compositions consist of the derivatives of benzoyl-methane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Advantageously, such combinations are combined with water-soluble filters such as, for example, 2-phenylbenzimidazole-5-sulfonic acid and their alkali metal, alkaline earth metal, ammonium, alkylammonium, alkanolammonium and glucammonium salts.

As well as said soluble substances, insoluble light protection pigments, namely finely dispersed metal oxides or salts, are also suitable for this purpose. Examples of suitable metal oxides are, in particular, zinc oxide and titanium oxide and also oxides of iron, zirconium, silicon, manganese, aluminum and cerium, and mixtures thereof. Salts which may be used are silicates (talc), barium sulfate or zinc stearate. The oxides and salts are used in the form of the pigments for skincare and skin-protective emulsions and decorative cosmetics. The particles here should have an average diameter of less than 100 nm, preferably between 5 and 50 nm and in particular between 15 and 30 nm. They can have a spherical shape, but it is also possible to use particles which have an ellipsoidal shape or a shape deviating in some other way from the spherical form. The pigments can also be surface-treated, i.e. hydrophilicized or hydrophobicized. Typical examples are coated titanium dioxides, such as, for example, titanium dioxide T 805 (Degussa) or Eusolex® T2000 (Merck). Suitable hydrophobic coating agents are here primarily silicones and, specifically in this case, trialkoxyoctylsilanes or simethicones. In sunscreens, preference is given to using micro- or nanopigments. Preference is given to using micronized zinc oxide. Further suitable UV light protection filters are given in the review by P. Finkel in SÖFW-Journal 122, 543 (1996) and Parf. Kosm. 3, 11 (1999).

As well as the two abovementioned groups of primary light protection substances, it is also possible to use secondary light protection agents of the antioxidant type; these interrupt the photochemical reaction chain which is triggered when UV radiation penetrates the skin. Typical examples thereof are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutic acid and derivatives thereof, α-glycosylrutin, ferulic acid, furfurylidene glucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

Biogenic Active Ingredients

Biogenic active ingredients are to be understood as meaning, for example, tocopherol, tocopherol acetate, tocopherol palmitate, ascorbic acid, (deoxy)ribonucleic acid and fragmentation products thereof, retinol, bisabolol, allantoin, phytantriol, panthenol, AHA acids, amino acids, ceramides, pseudoceramides, essential oils, plant extracts and vitamin complexes.

Deodorants and Antimicrobial Agents

Cosmetic deodorants counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents. Suitable antimicrobial agents are, in principle, all substances effective against gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorohexidine, 3,4,4′-trichlorocarbanilide (TTC), antibacterial fragrances, thymol, thyme oil, eugenol, oil of cloves, menthol, mint oil, farnesol, phenoxy-ethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen® CAT). The substances inhibit enzyme activity, thereby reducing the formation of odor. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, such as, for example, extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethol, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, camomile oil, oil of cloves, melissa oil, mint oil, cinnamon leaf oil, linden flower oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Antiperspirants reduce the formation of perspiration by influencing the activity of the eccrine sweat glands, thus counteracting underarm wetness and body odor. Aqueous or anhydrous formulations of antiperspirants typically comprise the following ingredients:

astringent active ingredients,

oil components,

nonionic emulsifiers,

coemulsifiers,

bodying agents,

auxiliaries, such as, for example, thickeners or complexing agents and/or

nonaqueous solvents, such as, for example, ethanol, propylene glycol and/or glycerol.

Suitable astringent antiperspirant active ingredients are primarily salts of aluminum, zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be:

anti-inflammatory, skin-protective or perfumed ethereal oils,

synthetic skin-protective active ingredients and/or

oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, such as, for example, xanthan gum, hydroxyethylcellulose, polyvinylpyrrolidone or high molecular weight polyethylene oxides.

Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof, and similar compounds.

Antidandruff Active Ingredients

Suitable antidandruff active ingredients are piroctone olamine (1-hydroxy-4-methyl-6-(2,4,4-trimethylpentyl)-2-(1H)-pyridinone monoethanolamine salt), Baypival® (climbazole) , Ketoconazole®, (4-acetyl-1-{-4-[2-(2,4-dichlorophenyl) r-2-(1H-imidazol-1-ylmethyl)-1,3-dioxylan-c-4-ylmethoxyphenyl}piperazine, ketoconazole, elubiol, selenium disulfide, colloidal sulfur, sulfur polyethylene glycol sorbitan monooleate, sulfur ricinol polyethoxylate, sulfur tar distillates, salicyclic acid (or in combination with hexachlorophene), undecylenic acid monoethanolamide sulfosuccinate Na salt, Lamepon® UD (protein undecylenic acid condensate), zinc pyrithione, aluminum pyrithione and magnesium pyrithione/dipyrithione magnesium sulfate.

Swelling Agents

The swelling agents for aqueous phases may be montmorillonites, clay mineral substances, Pemulen, and alkyl-modified Carbopol grades (Goodrich). Other suitable polymers and swelling agents are given in the review by R. Lochhead in Cosm. Toil. 108, 95 (1993).

Insect Repellents

Suitable insect repellents are N,N-diethyl-m-toluamide, 1,2-pentanediol or ethyl butylacetylaminopropionate.

Self-tanning Agents and Depigmentation Agents

A suitable self-tanning agent is dihydroxyacetone. Suitable tyrosine inhibitors, which prevent the formation of melanin and are used in depigmentation agents, are, for example, arbutin, ferulic acid, kojic acid, coumaric acid and ascorbic acid (vitamin C).

Hydrotropes

To improve the flow behavior, hydrotropes, such as, for example, ethanol, isopropyl alcohol, or polyols, can also be used. Polyols which are suitable here preferably have 2 to 15 carbon atoms and at least two hydroxyl groups. The polyols can also contain further functional groups, in particular amino groups, or be modified with nitrogen. Typical examples are

glycerol;

alkylene glycols, such as, for example, ethylene glycol, diethylene glycol, propylene glycol, butylene glycol, hexylene glycol, and polyethylene glycols with an average molecular weight of from 100 to 1 000 daltons;

technical-grade oligoglycerol mixtures with a degree of self-condensation of from 1.5 to 10, such as, for example, technical-grade diglycerol mixtures with a diglycerol content of from 40 to 50% by weight;

methylol compounds, such as, in particular, trimethylolethane, trimethylolpropane, trimethylolbutane, pentaerythritol and dipentaerythritol;

lower alkyl glucosides, in particular those with 1 to 8 carbon atoms in the alkyl radical, such as, for example, methyl and butyl glucoside;

sugar alcohols with 5 to 12 carbon atoms, such as, for example, sorbitol or mannitol,

sugars with 5 to 12 carbon atoms, such as, for example, glucose or sucrose;

amino sugars, such as, for example, glucamine;

dialcohol amines, such as diethanolamine or 2-amino-1,3-propanediol.

Preservatives

Suitable preservatives are, for example, phenoxyethanol, formaldehyde solution, parabenes, pentanediol or sorbic acid, and the other classes of substance listed in Annex 6, Part A and B of the Cosmetics Directive.

Perfume Oils

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pine wood, sandalwood, guaiac wood, cedarwood, rosewood), herbs and grasses (tarragon, lemon grass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. Preference is, however, given to using mixtures of different fragrances which together produce a pleasing fragrance note. Ethereal oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Dyes

Dyes which can be used are the substances which are approved and suitable for cosmetic purposes, as are summarized, for example, in the publication “Kosmetische Färbemittel” [Cosmetic Colorants] from the Farbstoffkommission der Deutschen Forschungs-gemeinschaft [Dyes Commission of the German Research Council], Verlag Chemie, Weinheim, 1984, pp. 81-106. These dyes are normally used in concentrations of from 0.001 to 0.1% by weight, based on the total mixture.

### EXAMPLES

### Example 1

### Extraction of the Plant Seeds

0.2 kg of defatted Argania spinosa seeds were transferred to a glass vessel and covered with 2 l of 80% strength by weight aqueous ethanol. This mixture was stirred at room temperature for two hours and the solids were separated off by means of filtration. The ethanol from the filtrate of the crude extract was removed under reduced pressure (15 to 20 torr). The remaining aqueous extract was extracted again with n-butanol. The butanol was likewise separated off under reduced pressure and the aqueous remainder was lyophilized. Based on the weight of the plant seeds, 0.5% by weight of saponins was obtained.

### Example 2

### Sensory Activity on Different Types of Hair

The evaluation of the modification of sensory properties on human hair in the comparison of damaged (bleached or permanently waved hair) with undamaged (control hair) after treatment with extracts of the plant Argania spinosa was carried out on standardized hair tresses (15 cm in length and 3 g in weight). The standard and placebo used was an aqueous sodium lauryl sulfate solution (15% w/v). The samples of the plant extracts were incorporated into the sodium lauryl sulfate solution in a concentration of 1.5% by weight and tested. The treatment effects were investigated on three different types of hair:

a) control hair: the tresses were washed with an aqueous sodium lauryl sulfate solution (15% by weight).

b) bleached hair: the tresses were treated with a bleaching shampoo which comprises 6% of H₂O₂ and ammonia (Éclair clair from L'Oréal) for 30 min and then washed with the aqueous sodium lauryl sulfate solution (15% by weight). This bleaching operation was repeated twice.

c) permanently waved hair: the tresses were treated with a sodium mercaptoacetate solution (6% w/v, pH=6) for 20 min, rinsed and then treated for 10 min with a solution of H₂O₂ (pH=3). After this solution had been mised out again, hair was washed with aqueous sodium lauryl sulfate solution (15% by weight). This permanent wave cycle was repeated twice.

The hair tresses prepared in this way were kept for 3 min in the solution with the respective test substance and then rinsed for 1 min. After rinsing, the tresses of hair were combed and their wet combability was tested. The tresses were dried at room temperature. The sensory tests were carried out on dry hair 24 h after treatment with the extracts.

The following properties were determined on the dry hair: combability, suppleness and softness, static charging, volume and shine.

The table gives the results of the sensory tests on wet and on dry hair. The sensory properties are to be read in comparison with standardized tresses of hair. The higher the number given, the better the evaluation of the respective sensory property.

TABLE 1
Sensory properties of human tresses of hair following
treatment with extracts of the plant Argania spinosa compared
with undamaged tresses of hair

| | Control | Bleached | Permanently |
|---|---|---|---|
| Parameter | hair | hair | waved hair |
| Combability on wet hair | 0 | 0.5 | 0 |
| Combability on dry hair | 0.5 | 0 | 0.5 |
| Suppleness and softness | 0.5 | 0.5 | 0.5 |
| Static charging | 0 | 0 | 0 |
| Volume | 0.5 | 0.5 | 0 |
| Shine | 0.5 | 0.5 | 0 |

The results of the test show that an extract of the plant Argania spinosa improves the sensory properties of human hair. There was no change in the static charging following treatment with the plant extract.

On bleached hair, an improvement in the combability of wet hair and in the volume and the shine were recorded. For permanently waved hair, the combability of dry hair and the softness increased. For the control hair, apart from the combability of wet hair and the static charging, a positive effect was recorded throughout. The static charging did not change for any of the types of hair used.

### Example 3

### Lipolysis Activity on Human Adipocytes

Background: Lipolysis is the name for the endogenous degradation of fats which are present in the adipocytes (fat cells) as reserves. These are cleaved enzymatically by lipases to give smaller molecular fragments the fatty acids and glycerol. The free fatty acids are then used by the muscle cells to produce energy.

Method: The adipocytes were isolated from human subcutaneous tissue, as corresponds to the general technique in accordance with Rodbell. The extract as in

Example 1 or the comparison substances are dissolved in the reference medium and then brought into contact with the isolated adipocytes for 90 minutes at 37° C. In each case one adipocyte preparations [sic] was analyzed. The percentage increase in released glycerol in the supernatant of the medium was determined by means of spectrophotometry in accordance with the method by Carpéné et al. The reference administered was the medium without substance to be analyzed and was set equal to 0.

TABLE 2
Lipolysis activity on human adipocytes

| | Concentra- | % increase |
|---|---|---|
| | tion in % | in released |
| Substance | by wt. | glycerol |
| Saponin extract as in Example 1 | 0.03 | 17 |
| Saponin extract as in Example 1 | 0.1 | 66 |
| Saponin extract as in Example 1 | 0.3 | 183 |
| Caffeine | 30 mM | 181 |

The results of the investigations show that the saponins from the seeds of Argania spinosa exhibit a lipolysis activity, dependent on the concentration, in human adipocytes in vitro. The results demonstrate the potential effect as slimming substances on the skin or the anticellulite action.

### Example 4

### Test on Human Fibroblasts Regarding Toxicity and Growth Factor Activity

The aim of these tests is to demonstrate a regenerating and revitalizing activity of extracts from Argania spinosa on human fibroblast cultures in vitro. The toxicity test permits the investigation of the concentration of test substance with which efficient test [sic] can be carried out. The toxicity and growth factor tests were carried out on human fibroblasts for investigating the regenerative activity of Argania spinosa extracts and for investigating a growth factor of comparable activity.

Method 1: Toxicity and cell growth test: Human fibroblasts were inoculated in a defined nutrient medium (DMEM=Dulbecco Minimum Essential Medium, Life Technologie Sarl) with 10% by weight of fetal calf serum and incubated for 24 h at 37° C. in a 5% strength CO₂ atmosphere. The nutrient medium with fetal calf serum was then replaced by a nutrient medium comprising DMEM without fetal calf serum. Varying concentrations of active substance in the form of the extracts from Argania spinosa as in Example 1 were added to this nutrient medium. For comparison, a test series of human fibroblasts without active substance was incubated as control. After incubation of the fibroblasts in the nutrient medium for three days, the growth and the metabolic activity was assessed by counting out the cells using a particle counter, and by determining the intracellular proportion of ATP in accordance with the Vasseur method (Journal francais Hydrologic, 1981, 9, 149-156) and the proportion of cell proteins in accordance with the Bradford method (Anal. Biochem., 1976, 72, 248-254). ATP or adenosine triphosphate is an energy transporter and is produced primarily by the mitochondria. The cells require ATP for the activity of many enzymes. The determination of the proportion of proteins gives an indication of the proportion of synthesized macromolecules such as enzymes, proteins, macromolecules of the dermis, which are necessary for the build-up and retention of the tissue.

TABLE 3
Toxicity test on human fibroblasts

| EC50 (in % by wt.) | Protein content | ATP content |
|---|---|---|
| Extract as in Example 1 | 0.0265% | 0.0204% |

The toxicity test shows that an extract as in Example 1 has an effective concentration (EC50 value) of 0.0265% by weight based on the protein content and a EC50 value of 0.0204% by weight based on the ATP content. These results show a low toxicity toward human fibroblasts in vitro.

TABLE 4
Increase in the protein content and the ATP content in human fibroblasts
following incubation with extracts as in Example 1

| | Concentration | Content of | Content of |
|---|---|---|---|
| Substance | in % by wt. | proteins in % | ATP in % |
| Control | | 100 | 100 |
| Extract as in Example 1 | 0.001 | 105 | 119 |
| | 0.003 | 113 | 113 |
| | 0.01 | 102 | 93 |

At concentrations of from 0.001 to 0.01% by weight of extract as in Example 1, an increase in the content of ATP of between 13 and 19% compared with the control was obtained. At concentrations of from 0.001 to 0.01% by weight of extract as in Example 1, the proportion of cell proteins increased by between 2 and 13% compared with the control.

Method 2: Improvement in the ability to survive The test [sic] were carried out on human fibroblasts. The test permits a certain number of parameters to be determined quantitatively on the inactive cells. The cultivation of the cells corresponds to the cultivation from Method 1, with the exception of the incubation time. The incubation time for these test [sic] were 72 h. The ability to survive was assessed by means of the proportion of MTT and the proportion of GSH. The mitochondrial activity is determined by MTT, a dye which is converted into formazane by the enzyme succinate dehydrogenase. This test was carried out in accordance with the Denizot F. and Lang R. method described in “Rapid calorimetric assay for cell growth and survival; J. Immunol. Methods, 89, 271-277, 1986”.

Glutathione (GSH) is a small peptide made of three amino acids which can protect the cell against oxidative stress or harmful environmental influences such as, for example, heavy metals. The proportion of GSH was carried out [sic] fluorometrically in accordance with the method by Hissin P J. and Hilf R. described in: “A fluorometric method for determination of oxydised and reduced Glutathione in tissus [sic]; Analytical Biochemistry, 74: 214-226, 1976”. The test [sic] were carried out three times and then repeated twice, thus giving six results per extract, which were averaged in each case. The results were determined as a percentage compared with the control.

TABLE 5
Parameters for determining the improvement
in the ability to survive

| | Concentration | Content | Content | |
|---|---|---|---|---|
| | of extract | of MTT | of protein | Content |
| | in % by wt. | in % | in % | of GSH |
| Control | 0 | 100 | 100 | 100 |
| Extract as | 0.001 | 106 | 96 | 119 |
| in Example 1 | 0.003 | 114 | 102 | 119 |
| | 0.001 | 110 | 87 | 184 |

The results show that an extract as in Example 1 from Argania spinosa markedly stimulates the synthesis of glutathione. This property enables the use of extracts from Argania spinosa as revitalizing and reactivating agents in cosmetic and/or pharmaceutical compositions. A use as skin-rejuvenating agent is likewise possible.

### Example 5

### Cell Protection Effect Against UVB on in vitro Cultivated Human Keratinocytes

Background: UVB rays trigger inflammation (erythema, odema) by activating an enzyme, namely phospholipase A2 or PLA2, which removes arachidonic acid from the phospholipids of the plasma membrane. Arachidonic acid is the precursor of the prostaglandins, which cause inflammation and cell membrane damage; the prostaglandins E2 (=PGE2) are formed by cyclooxygenase.

Method: The effect of UVB radiation was assessed on keratinocytes in vitro by determining the release of the cytoplasma enzyme LDH (lactate dehydrogenase). This enzyme serves as a marker for cell damage.

To carry out the tests, a defined medium, which comprises fetal calf serum, was inoculated with the keratinocytes, and the extract as in Example 1 (diluted with saline solution) was added 72 hours after the inoculation.

The keratinocytes were then irradiated with a UVB dose (30 mJ/cm²-tubes: DUKE FL40E).

After incubation at 37° C. and at 5% CO₂ for a further 1 day, the LDH content in the supernatant was determined. The content of LDH (lactate dehydrogenase) was determined by means of an enzyme reaction (kit used to investigate the LDH content from Roche). The number of adherent keratinocytes is determined (following trypsine treatment) using a particle-counting instrument.

TABLE 6
Cell protection effect of an extract of Argania spinosa against
UVB rays; results in % based on the control, average value
from 2 experiments, each with two repetitions

| Extract as in | Number of | Content of released |
|---|---|---|
| Example 1 | keratinocytes | LDH |
| Control without UV | 100 | 0 |
| UVB (30 mj/cm2 [sic]) | 49 | 100 |
| UVB + extract 0.03% | 90 | 44 |

The results of these tests demonstrate that an extract of the plant Argania spinosa according to the invention reduces the effect of UVB radiation on the number of keratinocytes and on the content of released LDH. Accordingly, the extracts described exhibit the ability to reduce the damage to cell membranes caused by UVB radiation.

### 6. Example

### Formulations of Cosmetic Compositions Comprising Argania spinosa Extracts

The saponins from Argania spinosa extract obtained according to Example 1 were used in the following inventive formulations K1 to K21 and also 1 to 25. The cosmetic compositions prepared in this way exhibited very good skincare properties coupled with good skin compatibility compared with the comparison formulations C1, C2 and C3. Moreover, the inventive compositions are stable against oxidative decomposition.

TABLE 7
Soft cream formulations K1 to K7
(All data in % by weight based on the cosmetic composition)

| INCI name | K1 | K2 | K3 | K4 | K5 | K6 | K7 | C1 |
|---|---|---|---|---|---|---|---|---|
| Glyceryl Stearate | 8.0 | 8.0 | 8.0 | 8.0 | 8.0 | 8.0 | 8.0 | 8.0 |
| (and) Ceteareth 12/20 | | | | | | | | |
| (and) Cetearyl | | | | | | | | |
| Alcohol (and) Cetyl | | | | | | | | |
| Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 3.0 | 3.0 | 3.0 | 3.0 | 3.0 | 3.0 | 3.0 | 3.0 |
| Glycerol (86% | 3.0 | 3.0 | 3.0 | 3.0 | 3.0 | 3.0 | 3.0 | 3.0 |
| strength by wt.) | | | | | | | | |
| Saponin extract as in | 0.5 | 0.5 | 0.5 | 0.5 | 0.5 | 0.5 | 0.5 | — |
| Example 1 | | | | | | | | |
| Tocopherol | | 0.5 | | | | | | |
| Allantoin | | | 0.2 | | | | | |
| Bisabolol | | | | 0.5 | | | | |
| Chitosan (Hydagen | | | | | 10.0 | | | |
| CMF) | | | | | | | | |
| Deoxyribonucleic | | | | | | 0.5 | | |
| acid¹⁾ | | | | | | | | |
| Panthenol | | | | | | | 0.5 | |

| | |
|---|---|
| Water | Ad 100 |

TABLE 8
Night cream formulations K8 to K14
(All data in % by weight based on the cosmetic composition)

| INCI name | K8 | K9 | K10 | K11 | K12 | K13 | K14 | C2 |
|---|---|---|---|---|---|---|---|---|
| Polyglyceryl-2 | 4.0 | 4.0 | 4.0 | 4.0 | 4.0 | 4.0 | 4.0 | 5.0 |
| Dipolyhydroxy- | | | | | | | | |
| stearate | | | | | | | | |
| Polyglyceryl-3 | 2.0 | 2.0 | 2.0 | 2.0 | 2.0 | 2.0 | 2.0 | 2.0 |
| Disostearate | | | | | | | | |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 8.0 | 8.0 | 8.0 | 8.0 | 8.0 | 8.0 | 8.0 | 8.0 |
| Dicaprylyl Ether | 5.0 | 5.0 | 5.0 | 5.0 | 5.0 | 5.0 | 5.0 | 5.0 |
| Magnesium Sulfate | 1.0 | 1.0 | 1.0 | 1.0 | 1.0 | 1.0 | 1.0 | 1.0 |
| Glycerol (86% | 5.0 | 5.0 | 5.0 | 5.0 | 5.0 | 5.0 | 5.0 | 5.0 |
| strength by wt.) | | | | | | | | |
| Saponin extract as in | 0.5 | 0.5 | 0.5 | 0.5 | 0.5 | 0.5 | 0.5 | — |
| Example 1 | | | | | | | | |
| Tocopherol | | 0.5 | | | | | | |
| Allantoin | | | 0.2 | | | | | |
| Bisabolol | | | | 0.5 | | | | |
| Chitosan (Hydagen | | | | | 10.0 | | | |
| CMF) | | | | | | | | |
| Deoxyribonucleic | | | | | | 0.5 | | |
| acid¹⁾ | | | | | | | | |
| Panthenol | | | | | | | 0.5 | |

| | |
|---|---|
| Water | Ad 100 |

TABLE 9
W/O body lotion formulations K15 to K21 (All data
in % by weight based on the cosmetic composition)

| INCI name | K15 | K16 | K17 | K18 | K19 | K20 | K21 | C3 |
|---|---|---|---|---|---|---|---|---|
| PEG-7 | 7.0 | 7.0 | 7.0 | 7.0 | 7.0 | 7.0 | 7.0 | 7.0 |
| Hydrogenated | | | | | | | | |
| Castor Oil | | | | | | | | |
| Decyl Oleate | 7.0 | 7.0 | 7.0 | 7.0 | 7.0 | 7.0 | 7.0 | 7.0 |
| Cetearyl | 7.0 | 7.0 | 7.0 | 7.0 | 7.0 | 7.0 | 7.0 | 7.0 |
| Isononanoate | | | | | | | | |
| Glycerol (86% | 5.0 | 5.0 | 5.0 | 5.0 | 5.0 | 5.0 | 5.0 | 5.0 |
| strength by wt.) | | | | | | | | |
| MgSO₄.7H₂O | 1.0 | 1.0 | 1.0 | 1.0 | 1.0 | 1.0 | 1.0 | 1.0 |
| Saponin extract | 1.5 | 1.5 | 1.5 | 1.5 | 1.5 | 1.5 | 1.5 | — |
| as in Example 1 | | | | | | | | |
| Tocopherol | | 0.5 | | | | | | |
| Allantoin | | | 0.2 | | | | | |
| Bisabolol | | | | 0.5 | | | | |
| Chitosan | | | | | 10.0 | | | |
| (Hydagen CMF) | | | | | | | | |
| Deoxy- | | | | | | 0.5 | | |
| ribonucleic | | | | | | | | |
| acid¹⁾ | | | | | | | | |
| Panthenol | | | | | | | 0.5 | |

| | |
|---|---|
| Water | Ad 100 |

¹⁾Deoxyribonucleic acid: molecular weight about 70,000, purity (determined by spectrophotometric measurement of the absorption at 260 nm and 280 nm): at least 1.7.

TABLE 10
Formulations

| | | | | | | |
|---|---|---|---|---|---|---|
| Composition (INCI) | 1% by wt. | 2% by wt. | 3% by wt. | 4% by wt. | 5% by wt. | 6% by wt. |

Cosmetic preparations conditioner (water, preservatives
ad 100% by weight)

| | | | | | | |
|---|---|---|---|---|---|---|
| Dehyquart ® A | 4.0 | 4.0 | | | 3.0 | |
| Cetrimonium Chloride | | | | | | |
| Dehyquart L ® 80 | | | 1.2 | 1.2 | | 1.0 |
| Dicocoylmethyl - | | | | | | |
| ethoxymonium | | | | | | |
| Methosulfate (and) | | | | | | |
| Propylene Glycol | | | | | | |
| Eumulgin ® B2 | 0.8 | | — | 0.8 | — | 1.0 |
| Ceteareth-20 | | | | | | |
| Eumulgin ® VL | — | 2.0 | 2.0 | — | 0.8 | — |
| 75 Lauryl Glucoside | | | | | | |
| (and) Polyglyceryl-2 | | | | | | |
| Polyhydroxy-stearate | | | | | | |
| (and) Glycerol | | | | | | |
| Lanette ® O | 3.0 | 3.0 | 3.0 | 3.0 | 3.0 | 3.0 |
| Cetearyl Alcohol | | | | | | |
| Cutina ® GMS | — | 0.5 | — | 0.5 | — | 1.0 |
| Glyceryl Stearate | | | | | | |
| Lamesoft ® PO 65 | | — | 3.0 | — | — | 3.0 |
| Cocoglucoside (and) | | | | | | |
| Glyceryl Oleate | | | | | | |
| Cetiol ® J 600 | — | 0.5 | — | 1.0 | — | 1.0 |
| Oleyl Erucate | | | | | | |
| Eutanol ® G | — | — | 1.0 | — | — | 1.0 |
| Octyldodecanol | | | | | | |
| Nutrilan ® Keratin W | 5.0 | — | — | 2.0 | — | — |
| Hydrolyzed Keratin | | | | | | |
| Generol ® 122 N | — | — | — | — | 1.0 | 1.0 |
| Soya Sterol | | | | | | |
| Saponin extract as | 1.0 | 1.0 | 1.0 | 1.0 | 1.0 | 1.0 |
| in Example 1 | | | | | | |
| Copherol ® 1250 | — | — | 0.1 | 0.1 | — | — |
| Tocopheryl Acetate | | | | | | |

| | 7% | 8% | 9% | 10% |
|---|---|---|---|---|
| Composition (INCI) | by wt. | by wt. | by wt. | by wt. |
| Cosmetic preparations conditioner (water, preservatives | | | | |
| ad 100% by weight) | | | | |
| Texapon ® NSO | 38.0 | 38.0 | 25.0 | — |
| Sodium Laureth Sulfate | | | | |
| Texapon ® SB 3 | — | — | 10.0 | — |
| Disodium Laureth | | | | |
| Sulfosuccinate | | | | |
| Plantacare ® 818 | 7.0 | 7.0 | 6.0 | — |
| Coco Glucosides | | | | |
| Plantacare ® PS 10 | — | — | — | 20.0 |
| Sodium Laureth Sulfate (and) | | | | |
| Coco Glucosides | | | | |
| Dehyton ® PK 45 | — | — | 10.0 | |
| Cocoamidopropyl Betaine | | | | |
| Lamesoft ® PO 65 | 3.0 | | | 4.0 |
| Coco-glucoside (and) Glyceryl | | | | |
| Oleate | | | | |
| Lamesoft ® LMG | — | 5.0 | — | |
| Glyceryl Laurate (and) | | | | |
| Potassium Cocoyl Hydrolyzed | | | | |
| Collagen | | | | |
| Euperlan ® PK 3000 AM | — | 3.0 | 5.0 | 5.0 |
| Glycol Distearate (and) | | | | |
| Laureth-4 (and) Cocamidopropyl | | | | |
| Betaine | | | | |
| Saponin extract as in | 1.0 | 1.0 | 1.0 | 1.0 |
| Example 1 | | | | |
| Arlypon ® F | 3.0 | 3.0 | 1.0 | — |
| Laureth-2 | | | | |
| Sodium chloride | — | 1.5 | — | 1.5 |

| Composition (INCI) | 11 | 12 | 13 | 14 |
|---|---|---|---|---|
| Cosmetic preparations “two in one” shower preparation | | | | |
| (water, preservatives ad 100% by weight) - continuation | | | | |
| Texapon ® NSO | 30.0 | 25.0 | | 25.0 |
| Sodium Laureth Sulfate | | | | |
| Plantacare ® 818 | | | | 8.0 |
| Coco Glucosides | | | | |
| Plantacare ® 2000 | | 8.0 | | |
| Decyl Glucoside | | | | |
| Plantacare ® PS 10 | | | 20.0 | |
| Sodium Laureth Sulfate (and) | | | | |
| Coco Glucosides | | | | |
| Dehyton ® PK 45 | | 10.0 | 10.0 | |
| Cocoamidopropyl Betaine | | | | |
| Lamesoft ® PO 65 | 5.0 | | | |
| Coco-glucoside (and) Glyceryl | | | | |
| Oleate | | | | |
| Lamesoft ® LMG | | 5.0 | 5.0 | |
| Glycerol Laurate (and) | | | | |
| Potassium Cocoyl Hydrolyzed | | | | |
| Collagen | | | | |
| Gluadin ® WQ | 3.0 | | | |
| Laurdimonium Hydroxapropyl | | | | |
| Hydrolyzed Wheat Protein | | | | |
| Gluadin ® WK | | | | |
| Sodium Cocoyl Hydrolyzed Wheat | | | | |
| Protein | | | | |
| Euperlan ® PK 3000 AM | 5.0 | 3.0 | 4.0 | — |
| Glycol Distearate (and) | | | | |
| Laureth-4 (and) Cocamidopropyl | | | | |
| Betaine | | | | |
| Panthenol | 0.5 | — | — | 0.5 |
| Saponin extract as in Example 1 | 1.0 | 1.0 | 1.0 | 1.0 |
| Arlypon ® F | 2.6 | 1.6 | — | 1.0 |
| Laureth-2 | | | | |
| Sodium chloride | — | — | — | — |

| Composition (INCI) | 15 | 16 | 17 | 18 | 19 | 20 |
|---|---|---|---|---|---|---|
| Cosmetic preparations shampoo (water, preservatives ad | | | | | | |
| 100% by weight) - continuation | | | | | | |
| Texapon ® NSO | 30.0 | | | 30.0 | 25.0 | |
| Sodium Laureth Sulfate | | | | | | |
| Texapon ® K 14 S | | 30.0 | | | | 30.0 |
| Sodium Myreth Sulfate | | | | | | |
| Texapon ® SB 3 | | 10.0 | | | | |
| Disodium Laureth | | | | | | |
| Sulfosuccinate | | | | | | |
| Plantacare ® 818 | 4.0 | | | | | |
| Coco Glucosides | | | | | | |
| Plantacare ® 2000 | | 4.0 | | | | |
| Decyl Glucoside | | | | | | |
| Plantacare ® PS 10 | | | 20.0 | | | |
| Sodium Laureth Sulfate | | | | | | |
| (and) Coco Glucosides | | | | | | |
| Dehyton ® PK 45 | 5.0 | | | 10.0 | | 10.0 |
| Cocoamidopropyl | | | | | | |
| Betaine | | | | | | |
| Gluadin ® WK | | | | | 8.0 | |
| Sodium Cocoyl | | | | | | |
| Hydrolyzed Wheat | | | | | | |
| Protein | | | | | | |
| Lamesoft ® PO 65 | — | — | — | — | 2.0 | 2.0 |
| Coco-glucoside (and) | | | | | | |
| Glyceryl Oleate | | | | | | |
| Nutril ® Keratin W | 5.0 | — | — | — | | — |
| Hydrolyzed Keratin | | | | | | |
| Gluadin ® W 40 | — | 2.0 | — | 2.0 | — | — |
| Hydrolyzed Wheat | | | | | | |
| Protein | | | | | | |
| Euperlan ® PK 3000 AM | — | — | — | 3.0 | 3.0 | — |
| Glycol Distearate | | | | | | |
| (and) Laureth-4 (and) | | | | | | |
| Cocamidopropyl Betaine | | | | | | |
| Panthenol | — | — | — | — | — | 0.2 |
| Saponin extract as in | 1.0 | 1.0 | 1.0 | 1.0 | 1.0 | 1.0 |
| Example 1 | | | | | | |
| Arlypon ® F | 1.5 | — | — | — | — | — |
| Laureth-2 | | | | | | |
| Sodium chloride | — | 1.6 | 2.0 | 2.2 | — | 3.0 |

| Composition | | | | | |
|---|---|---|---|---|---|
| Name according to INCI | 21 | 22 | 23 | 24 | 25 |
| Cosmetic preparations foam bath (water, preservatives | | | | | |
| ad 100% by weight) - continuation 2 | | | | | |
| Texapon ® NSO | — | 30.0 | 30.0 | — | 25.0 |
| Sodium Laureth Sulfate | | | | | |
| Plantacare ® 818 | — | 10.0 | — | — | 20.0 |
| Coco Glucosides | | | | | |
| Plantacare ® PS 10 | 22 .0 | — | 5.0 | 22.0 | — |
| Sodium Laureth Sulfate (and) | | | | | |
| Coco Glucosides | | | | | |
| Dehyton ® PK 45 | 15.0 | 10.0 | 15.0 | 15.0 | 20.0 |
| Cocoamidopropyl Betaine | | | | | |
| Monomuls ® 90-O 18 | 0.5 | | | | |
| Glyceryl Oleate | | | | | |
| Lamesoft ® PO 65 | | 3.0 | | 3.0 | |
| Coco-glucoside (and) Glyceryl | | | | | |
| Oleate | | | | | |
| Cetiol ® HE | | | 2.0 | | 2.0 |
| PEG-7 Glyceryl Cocoate | | | | | |
| Nutrilan ® 1-50 | 5.0 | | | | |
| Hydrolyzed Collagen | | | | | |
| Gluadin ® W 40 | | 5.0 | | 5.0 | |
| Hydrolyzed Wheat Gluten | | | | | |
| Gluadin ® WK | | | | 7.0 | |
| Sodium Cocoyl Hydrolyzed | | | | | |
| Wheat Protein | | | | | |
| Euperlan ® PK 3000 AM | 5.0 | — | — | 5.0 | — |
| Glycol Distearate (and) | | | | | |
| Laureth-4 (and) | | | | | |
| Cocamidopropyl Betaine | | | | | |
| Arlypon ® F | | | 1.0 | | |
| Laureth-2 | | | | | |
| Sodium chloride | 1.0 | | 1.0 | | 2.0 |
| Saponin extract as in | 1.0 | 1.0 | 1.0 | 1.0 | 1.0 |
| Example 1 | | | | | |

(1-4) Hair rinse,
(5-6) hair cure
(7-8) shower preparation,
(9) shower gel,
(10) washing lotion

1. A cosmetic and/or pharmaceutical preparation that contains saponins from an extract of the plant Argania spinosa.

2. The preparation as claimed in claim 1, characterized in that the extract is obtained by extraction from parts of plants, selected from the group consisting of the leaves, the roots, the stem, the bark, the flowers, the fruits, the fruit flesh and the seeds.

3. The preparation as claimed in claim 1, characterized in that the extract is obtained by extraction of the seeds and/or the defatted seeds from the fruit of the plant.

4. The preparation as claimed in claim 1, characterized in that it comprises saponins selected from the group consisting of arganine A, arganine B, arganine C, arganine D, arganine E, arganine F, arganine G, arganine H, arganine J and misaponine A.

5. The preparation as claimed in claim 1, characterized in that it comprises the plant extract containing the saponines in amounts of from 0.01 to 25% by weight, based on the final preparation, with the proviso that the quantitative data add up to 100% by weight with water and optionally further auxiliaries and additives.

6. The use of preparations as claimed in any of claims 1 to 5 in care compositions for hair and/or skin.

7. The use of preparations as claimed in claim 6 for improving the combability, the shine, the volume and the flexural strength of hair.

8. The use of preparations as claimed in claim 6 as slimming and anticellulite care compositions for the skin.

9. The use of preparations as claimed in claim 6 as skincare compositions with antirosacea effect.

10. The use of preparations as claimed in claim 6 in protective and restorative care compositions for stimulating the metabolism and the immune defense of the skin and of the hair follicles with revitalizing and reactivating activities for the skin and the hair follicles.

11. The use of preparations as claimed in claim 6 in sunscreens.
